(19) United States
(12) Patent Application Publication
(10) Pub. No.: US 2008/0319247 A1
(43) Pub. Date: Dec. 25, 2008

(54) Method of local therapy using magnetizable thermoplastic implant

(75) Inventors: Zachary Graham Forbes, Salt Lake City, UT (US); Steven Michael Kurtz, Haddonfield, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Philadelphia Health & Education Corporation d/b/a Drexel University College of Medicine, Philadelphia, PA (US)

(21) Appl. No.: 12/214,647

(22) Filed: Jun. 20, 2008

Related U.S. Application Data
(60) Provisional application No. 60/945,370, filed on Jun. 21, 2007.

(51) Int. Cl. A61N 2/00; A61F 2/02; A61H 1/00; A61B 19/00

(52) U.S. Cl. 600/9; 623/11.11; 601/15; 128/898

(57) ABSTRACT

A method for local therapies using heat generating implants comprising magnetic or magnetizable features or objects distributed in a solidified moldable matrix to treat bone infection or loosening of implants by the mechanism of hyperthermia or thermoablation.

### RELATED APPLICATIONS

This application claims the benefit of U.S. Provisional Application Ser. No. 60/945,370, filed Jun. 21, 2007. The entire contents of this application is incorporated herein by this reference.

### BACKGROUND OF THE INVENTION

Magnetic implants combined with magnetic fields to target drugs in the body have been previously described (see, for example U.S. Patent Application Publications No. 2006/0041182A1 to Forbes et al. and 2006/0025713 to Rosengart et al.).

Following confirmation of its biocompatibility two decades ago, polyaryletherketones (PAEKs) have been increasingly employed as biomaterials for orthopedic, trauma, and spinal implants. Commercialized for industry in the 1980s, PAEK is a relatively new family of high temperature thermoplastic polymers, consisting of an aromatic backbone molecular chain, interconnected by ketone and ether functional groups. Three PAEK polymers, used previously for orthopedic and spinal implants, include poly(aryl-ether-ketone) (PEK), poly(aryl-ether-ether-ketone) (PEEK), and poly(aryl-ether-ketone-ether-ketone-ketone (PEKEKK). The chemical structure of polyaromatic ketones confers stability at high temperatures (exceeding 300° C.), resistance to chemical and radiation damage, compatibility with many reinforcing agents (such as glass and carbon fibers), and greater strength (on a per mass basis) than many metals, making it highly attractive in industrial applications, such as, for example, aircraft and turbine blades.

Historically, the availability of polyaromatic polymers arrived at a time when there was growing interest in the development of isoelastic hip stems and fracture fixation plates, with stiffnesses comparable to bone. Although neat (unfilled) polyaromatic polymers can exhibit an elastic modulus ranging between 3-4 GPa, the modulus can be tailored to closely match cortical bone (18 GPa) or titanium alloy (110 GPa) by preparing carbon fiber composites with varying fiber length and orientation. In the 1990s, researchers characterized the biocompatibility and in vivo stability of various PAEK materials, along with other high performance engineering polymers, such as polysulphones and polybutylene terephthalate. However, concerns were raised about the stress-induced cracking of polysulphones following polysulphones, and use of these polymers in implants was subsequently abandoned. Other polyaromatic ketone polymers, such as PEK and PEKEKK, were discontinued by their industrial supplier and thus ceased to be available for biomaterial applications.

By the 1990s, PEEK had emerged as the leading high performance thermoplastic candidate for replacing metal implant components, especially in orthopedics and trauma. Not only was the material resistant to simulated in vivo degradation, including damage caused by lipid exposure, but starting in April 1998 PEEK was offered commercially as a biomaterial for implants (Invibio, Ltd.: Manchester, United Kingdom). Facilitated by a stable supply, research on PEEK biomaterials flourished and is expected to continue to advance in the future.

Numerous studies documenting the successful clinical performance of polyaryletherketone polymers in orthopedic and spine patients continue to emerge in the literature. Recent research has also investigated PEEK composites as bearing materials and flexible implants used for joint arthroplasty. Due to interest in further improving implant fixation, PEEK biomaterials research has also focused on compatibility of the polymer with bioactive materials, including hydroxyapatite, either as a composite filler, or as a surface coating. As a result of ongoing biomaterials research, PEEK and related composites can be engineered today with a wide range of physical, mechanical, and surface properties, depending upon their implant application.

The versatility of PEEK biomaterials necessarily translates into increased complexity, both for implant designers, as well as for researchers seeking to explore new modifications of PEEK for novel implant applications. In recent years, advances in the processing and biomaterials applications of PEEK have been progressing steadily.

Hyperthermia has been proposed as a means for cancer treatment by the use of alternating magnetic fields to heat particles concentrated in or around a tumor. Localized hyperthermia technique using magnetic particles, based on proposal brought forward by Gilchrist in 1957. It has been found that the viability of cancer cells is reduced and their sensitivity to chemotherapy and radiation increase when the human or animal malignant cells are heated to temperatures between 41-46° C. Magnetic hyperthermia provides the heat at the site of concentration invasively by applying an external alternating magnetic field to the magnetic particles. The particles will heat up and conduct the heat to the local area. The use of materials with Curie temperature in the range of 41-46° C. is desired to provide a safeguard against overheating of normal cells, due to the decrease of magnetic coupling in the paramagnetic regime (above Tc). However, temperatures just above 37° C. and higher may be used for ablation or denaturing of bacteria, fungal, or other microbial contamination at the site of magnetic particle concentration. Magnetic nanoparticles are typically used in the study of treatment of cancers, primarily for their ability to maneuver the vasculature to localize at or within tumors, while minimizing systemic distribution. If certain ferromagnetic or paramagnetic materials are concentrated within the matrix of a device implanted within the body, concerns of biocompatibility are less if these materials are firmly oriented within the matrix or structure of the implant. This allows the use of magnetic particles or features from the nanometer up to millimeters in diameter, as these particles are encased within a solid matrix and may not deviate into the vascular system. This is beneficial for adaptability to different applications, cost/benefit, biocompatibility issues, material processing, and other.

International Application Publication No. WO/9725062 to Kaiser et al. describes magnetic material for hyperthermic tumor therapy which comprises suspension of iron oxide particles generating heat on application of alternating magnetic field.

Takegami et al. describe the use of ferromagnetic bone cement as a thermoseed to generate heat for treatment of bone tumors (Takegami et al., 1998 J Biomed Mater Res 43:210-214).

Despite current developments, there is a need in the art to provide methods and materials for treatment of infections or loosening of implants.

### BRIEF SUMMARY OF THE INVENTION

This invention provides a new method for local therapies using heat generating implants comprising magnetic or magnetizable features or objects distributed in a solidified moldable matrix o treat bone infection or loosening of implants by the mechanism of hyperthermia or thermoablation.

Currently used implants (e.g., cardiovascular, orthopedic, etc.) can be modified by addition of magnetic or magnetizable objects, which enable the treatment of infection or other local complications by heating of the implant.

This invention is particularly useful for orthopedic applications and is intended to treat local or generalized infections of bone and bone marrow typically caused by bacteria introduced from trauma, surgery, implant use, by direct colonization from a proximal infection or via systemic circulation. This invention can also be used, for example, for treatment of bone degeneration due to aseptic loosening or other complications involving orthopedic implants. Other applications of the invention include dental procedures. Generally, any procedures which involve placing an implant in a body can benefit from the present invention.

Conventional therapy which uses systemic antibiotics is expensive, prone to complications and often unsuccessful. High systemic dosage of antibiotics to facilitate sufficient tissue and biofilm penetration is not preferable because of possible serious toxic side effects. As a result, many chronic infections as well as implant loosening require revision surgeries.

Accordingly, in one aspect, the invention is heat producing implant comprising a solidified product of a moldable matrix having magnetic or magnetizable objects distributed in a pattern such that at least 50% of the magnetic or magnetizable objects are oriented along the surface of the implant, wherein the heat producing implant is capable of generating a controllable heat upon application of controlled alternating magnetic fields in the intensity sufficient to destroy infection producing microorganisms.

In another aspect, the invention is a method of preventing or eliminating an infection of an internal cavity of a mammal in the proximity of an implant, the method comprising: providing a moldable matrix; providing magnetic/magnetizable objects; combining the magnetic/magnetizable objects with the moldable matrix to form a composite and optionally orienting the magnetic/magnetizable objects within the moldable matrix; solidifying the composite and thereby forming a heat producing implant; administering the heat producing implant to the internal cavity of the mammal; activating the heat producing implant to prevent or eliminate the infection, wherein said activating is produced by applying an alternating magnetic field to the magnetic/magnetizable objects in the intensity sufficient to prevent or destroy infection producing microorganisms.

This invention provides noninvasive treatment of infections and loosening using heat for local hyperthermia or thermoablation. The invention has the following additional advantages: procedures are repeatable for the lifetime of the implant, and magnetizable thermoplastic joint stems can be tailored to meet strict mechanical needs using, for example, glass or carbon coated magnetite.

### DETAILED DESCRIPTION

The object of the invention is to provide a long term, repeatable, noninvasive treatment for infections and local pathology around an implant. Sufficient doses of anti-microbial, anti-inflammatory or other drugs are difficult to concentrate at implant surfaces, particularly in orthopedic implants, and in many cases may only deliver such a small concentration that resistant bacteria emerge, increasing the odds of chronic infection. This invention offers a noninvasive approach to treating complications at the implant site, reducing the occurrence of acute or chronic infections, as well as reducing the need for revision surgery by using an implant to generate a localized heat at a place of the infection or other pathology. In addition, by integrating the magnetic material into a thermoplastic implant, instead of using a metallic implant as are currently used, leaching of metallic ions can be eliminated or reduced.

The inventors have discovered that a heat producing implant (e.g., a joint replacement, a pin, a screw, or a plate) can be made by distributing paramagnetic, superparamagnetic, or ferromagnetic material within the matrix of a thermoplastic implant, which can then be activated as needed. Accordingly, the thermogenic implant of the invention is made from a moldable matrix having magnetic/magnetizable objects distributed in a pattern such that at least 50% of the magnetic or magnetizable objects are oriented along the surface of the implant, wherein the heat producing implant is capable of generating a controllable heat upon application of controlled alternating magnetic fields in the intensity sufficient to destroy infection producing microorganisms.

After these implants are placed in the body, magnetic fields can be used to heat the implant in order to eradicate microbial infections, or in cases of loosening, to necrose tissue with the intent of inducing rapid scarring and subsequent re-integration of the implant into the surrounding tissue. The application of alternating magnetic fields produces a controllable heating at the outer surface of the implant which comes in contact with a tissue or a bone and thereby facilitates local hyperthermia and thermoablation.

Non-limiting examples of orthopedic implants, which can be fabricated from this material include screws, bone plates, pins, bone prosthesis, etc. Other types of implants such as vascular stents, prostate seeds, hernia meshes, and endovascular catheter devices can be inserted temporarily to perform hyperthermia or thermoablation procedures.

In certain embodiments, implants of the invention can be provided with a functionalized coating for various purposes such as, for example, a coating with a bioactive material to confer desired properties (e.g., a drug, a growth factor, a cell, hydroxyapatite, etc.), an antimicrobial coating (e.g., with an antibiotic or silver coating) to decrease initial surface antimicrobial activity, etc.

### Magnetic of Magnetizable Objects

By the term “magnetic or magnetizable object”, as used herein, it is meant a particle, an object having a variety of geometric shapes (e.g., a rod, a disk, a wire, a mesh, etc.) or a surface coating on a preexisting device, which are made from materials that strongly conduct magnetic flux. The terms “magnetic” or “magnetizable” are used interchangeable herein. Magnetic or magnetizable objects may comprise glass or carbon coated magnetic or magnetizable nano or microspheres, or carbon or glass rods containing segmentations of magnetic or magnetizable material. These materials can be oriented during the molding process. The magnetic or magnetizable objects are non-biodegradable and are preferably permanently embedded within the implant, on the outer surface thereof or arranged in a combination of the above variants. The magnetic or magnetizable objects can be mixed with an implant matrix prior to its solidifying or added at the time of solidification. If a magnetic or magnetizable surface coating is required, such coating can be added to the surface of the preexisting device or added to a device fabricated with the embedded magnetic or magnetizable objects.

The magnetizable carrier or particle of the invention can be prepared by methods known in the art in various shapes and sizes (see, for example Hyeon T., Chemical Synthesis of Magnetic Nanoparticles. The Royal Society of Chemistry 2003, Chem. Commun., 2003, 927-934). In certain embodiments, iron oxide nanocrystals were obtained by precipitation of mixed iron chlorides in the presence of a base in aqueous medium (see Khalafalla S E. Magnetic fluids, Chemtech 1975, September: 540-547).

Magnetizable carriers can be in a shape of particles, crystals, spheres, rods, wires, blocks, pellets, or other dispersions. Magnetizable materials are added to the curable matrix of the invention (e.g., bone cement) to make it magnetizable.

In certain embodiments of the method, the magnetizable carrier is a magnetizable particle with a diameter from about 10 nm to about 1000 nm. Preferably, the magnetizable particle has a diameter from 10 nm to 500 nm.

Exemplary magnetizable particles Spherotech (Spherotech, IL) have 20% γ-Fe₂O₃ magnetite by weight a nominal diameter of 350 nm with approximately 10% variance in size. These particles have a carboxylate per nm² of surface area, which can be used as a linker for bioactive or diagnostic agents with corresponding reactive functional groups.

Those skilled in the art would be able to select material for making the magnetizable carrier or particle such that it would be magnetized in the presence of an external magnetic field as those materials are known or are being developed (e.g., metals, metal alloys and rear earth elements).

The magnetic material suitable for this invention may consist of one or a combination of paramagnetic, superparamagnetic, ferromagnetic, or rare earth metal permanent magnets. The size of the magnetic or magnetizable objects may range from nanometers to centimeters in diameter. Geometry of the magnetic or magnetizable objects can vary depending on applications. The distribution of the magnetic or magnetizable objects in an implant can vary; for example, the magnetic or magnetizable objects may be uniformly distributed throughout the thermoplastic, and/or oriented specifically along the inside or outside perimeter to maximize heating. Examples of magnetic or magnetizable materials useful in the present invention include, but are not limited to, cobalt, iron, iron oxides, nickel, manganese, and rare earth magnetic materials (e.g., samarium and neodymium) and various soft magnetic alloys (e.g., Ni—Co). In one embodiment, the magnetizable object is magnetized only in the presence of externally applied magnetic fields.

Parameters of heat generated by the implant (temperature, intensity, depth of penetration, etc.) can be controlled by the concentration, geometry and size of magnetic objects in the implant, as well as the frequency of the magnetic field.

Those skilled in the art would be able to select material for making the magnetizable objects such that they would be magnetized in the presence of an external magnetic field as those materials are known or are being developed (e.g., metals, metal alloys and rear earth elements). In certain embodiments, the magnetizable object is made from at least one of cobalt, nickel, iron, manganese, samarium and neodymium.

An implant can be made magnetizable by coating a preexisting device with a magnetizable coating by methods known in the art such as, for example, electrodeposition or electrospraying.

The term “coating”, as used herein, includes coatings that completely cover an outer surface of the implant, or a portion thereof (e.g., continuous coatings, including those that form films on the surface), as well as coatings that may only partially cover the outer surface of the implant, such as those coatings that after drying leave gaps in coverage on a surface (e.g., discontinuous coatings). The later category of coatings may include, but is not limited to a network of covered and uncovered portions. Coatings can be flat or raised above the surface or embossed on the surface (e.g., a ridge) or it can be in a shape of dots or other shapes creating a pattern. A combination of various coatings can also be used.

Coating can be made from a magnetizable material (e.g., stainless steel, soft magnetic alloys) and a non-magnetizable material (a polymer). Selecting the appropriate combination of coating and support materials, it is desirable that the magnetizable object is prepared based on the selection may have a set of segments on its surface that will enable the creation of a localized magnetic gradient. For example, if the support is made from a magnetizable compound, material(s) of the segment can have a higher or a lower degree of magnetization or they can be made from non-magnetizable materials. On the other hand, if the support or a surface of the magnetizable object is made from a non-magnetizable compound, material(s) of the segment must be made from a magnetizable compound.

The magnetic particles may be encapsulated within a biological or pharmaceutical polymer, such as, for example, dextran, or poly (lactic glycolic) acid (PLGA), or other biodegradable material. Encapsulated particles or clumps of magnetic material can be bound with or encapsulate bioactive agents (e.g., antibiotics, antiseptics, radioactive agents, biological cells, anti-neoplastics, anti-inflammatories, mitogenic drugs, morphogenic drugs, or other therapeutic agents) or diagnostic agents.

### Implant Moldable Matrix

The implants of the invention comprise a solidified product of a moldable matrix which hosts magnetic/magnetizable objects.

In a preferred embodiment, the moldable matrix is a thermoplastic polymer such as, for example, polyaryletherketones (PAEKs), polyethylenes, polyurethanes. Non-limiting examples of PAEKs include poly(aryl-ether-ketone) (PEK), poly(aryl-ether-ether-ketone) (PEEK), and poly(aryl-ether-ketone-ether-ketone-ketone (PEKEKK).

Although the preferred embodiment is a neat PAEK thermoplastic, those skilled in the art will recognize that the method may be practiced with the much broader range of thermoplastic materials with a melt transition above body temperature, including PAEK composites containing other fillers, such as radiopacifiers and carbon fibers, as well as ultra-high molecular weight polyethylene, high density polyethylene, polybutylene terepthalate, polysulfone, and polyurethane.

There are two main routes involved in the production of PAEKS. The first method involves linking aromatic ether species through ketone groups, whilst a second method involves linking aromatic ketones by an ether bond. The first method involves an electrophillic reaction and Friedel Crafts acylation chemistry whilst the second route involves a nucleophillic displacement reaction.

PEEK represents the dominant member of the PAEK polymer, and can be processed using a variety of commercial techniques, including injection molding, extrusion and compression molding, at temperatures between 390° C. and 420° C. At room and body temperature, PEEK is in its “glassy” state, as its glass transition temperature occurs about 143° C., whereas the crystalline melt transition temperature (Tₘ) occurs around 343° C. After polymerization, PEEK is chemically inert and insoluble in all conventional solvents at room temperature, with the exception of 98% sulphuric acid.

The literature on PAEK resin is a maze of trade names and producers, which have changed over the years, complicating interpretation of published data for today's materials. For researchers interested in deciphering the historical polymer science literature, we provide here a brief primer on the nomenclature of PAEK resins used for industrial purposes as well as for biomaterials (Table 1). Resin, when used in this context, refers to the neat, unfilled powder that is created by polymerization, whereas grades are typically characterized by flow characteristics (e.g., for injection molding or compression molding) or based on their filler content (e.g., glass fiber or carbon fiber). Because PAEK polymers are converted using standard thermoplastic processing techniques, such as injection molding, they are generally available as pellets, although powder resin is also available. Stock shapes, such as rods, are also available from producers.

TABLE 1
Summary of PAEK Materials Used for Implants

| Polymer | Trade Name | Producer |
|---|---|---|
| PEEK | OPTIMA (Biomaterial) | Invibio (formerly Victrex) |
| | | Manchester, UK |
| PEEK | Victrex | Victrex, Manchester, UK |
| PEEK | Gatone | Gharda, India |
| PEEK | Keto-Spire | Solvay Advanced Polymers, LLC |
| PEK | PEK | Victrex |
| PEKK | PEKK | DuPont (Wilmington, DE) |
| PEKEKK | Ultrapek | BASF, United States |

TABLE 2
Typical Average Physical Properties of PEEK and PEEK structural composite
biomaterials, compared with ultra-high molecular weight polyethylene (UHMWPE) and
polymethyl methacrylate (PMMA)

| | Selected Invibio PEEK Biomaterials | | | | |
|---|---|---|---|---|---|
| | (OPTIMA LT1) | | | | |
| | | 30% (w/w) | 68% (v/v) | | |
| | | Chopped | Continuous | | |
| | | Carbon | Carbon | | |
| | Unfilled | Fiber | Fiber | | |
| | (OPTIMA | Reinforced | Reinforced | | |
| Property (ISO) | LT1) | (LT1CA30) | (Endolign) | UHMWPE | PMMA |
| Polymer Type | Semi-crystalline | | | Semi- | Amorphous |
| | | | | crystalline | |
| Molecular Weight | 0.08-0.12 | 0.08-0.12 | 0.08-0.12 | 2-6 | 0.1-0.8 |
| (10⁶ g/mole) | | | | | |
| Poisson's ratio | 0.36 | 0.40 | 0.38 | 0.46 | 0.35 |
| Specific gravity | 1.3 | 1.4 | 1.6 | 0.932-0.945 | 1.180-1.246 |
| Flexural | 4 | 20 | 135 | 0.8-1.6 | 1.5-4.1 |
| Modulus (GPa) | | | | | |
| Tensile | 93 | 170 | >2000 | 39-48 | 24-49 |
| Strength (MPa) | | | | | |
| Tensile | 30-40 | 1-2 | 1 | 350-525 | 1-2 |
| elongation (%) | | | | | |
| Degree of | 30-35 | 30-35 | 30-35 | 39-75 | Noncrystalline |
| crystallinity (%) | | | | | |

Testing conducted at 23° C.

Thermoplastic polymers, including polyaryletherketones (PAEKs) such as PEEK, are a broad class of materials that are processed by heating up the polymer, forming the molten polymer into a desired shape, and then cooling back down to room temperature. Certain thermoplastics, while in the molten state, are introduced into a mold and then cooled, in a process known as injection molding. For other applications, the molten thermoplastic is extruded into its final shape, blow molded, or compression molded.

In certain embodiments, the moldable matrix is a bone cement. In certain embodiments, commercially produced bone cement made of polymethyl methacrylate or hydroxyapatite, or otherwise polymerizable bone cement material.

The term “bone cement” as used herein, includes any suitable bone cement useful in orthopedic or dental applications. Exemplary bone cements include those described by U.S. Pat. No. 6,593,394 to Li et al and U.S. Pat. No. 5,336,700 to Murray, which are incorporated herein in their entireties.

In orthopedics, an acrylate (e.g., polymethylmethacrylate (PMMA)) based bone cement is used to affix implants and to remodel lost bone. It is supplied as a powder with liquid methyl methacrylate (MMA). When mixed together, PMMA and MMA yield a dough-like cement that gradually hardens in the body. Surgeons can judge the curing of the PMMA bone cement by the smell of MMA in the patient's breath. Although PMMA is biologically compatible, MMA is considered to be an irritant and a possible carcinogen. PMMA has also been linked to cardiopulmonary events in the operating room due to hypotension.

The powder used in making the cement typically includes fine particles of polymethylmethacrylate (PMMA), polymethylmethacrylate styrene co-polymer, and benzoyl peroxide. Barium sulfate is optionally added to provide X-ray opacity and may constitute approximately 10 percent by weight of the powder. The benzoyl peroxide acts as a chemical initiator and may constitute approximately 2 percent by weight of the cement powder. The cement powder is primarily very small rounded particles of PMMA and PMMA styrene co-polymer. Orthopedic cement powder also includes exceedingly fine particles of PMMA and PMMA styrene co-polymer. Dental cement powder typically does not include the exceedingly fine particles.

The methylmethacrylate (MMA) monomer liquid mixed with the cement powder typically includes dimethyl-p-toluidine and hydro-quinone. The dimethyl-p-toluidine is a cold-curing agent which may constitute approximately 2.6 percent by weight of the liquid. The hydroquinone is a stabilizer usually added in very small amounts.

Loose PMMA cement powder is mixed directly with the MMA monomer liquid in a ratio of approximately 40 grams of powder to 20 ml. of liquid. Mixed cement is usable for approximately 10 minutes after the start of mixing. The short useful life of the cement places a premium on rapid mixing of the cement and delivering the cement to the application site.

Both the liquid and powder components may contain the conventional additives in this field. Thus, for example, the powder component may contain minor amounts of an X-ray contrast material, polymerization initiators and the like. The liquid component may contain crosslinking agents and minor amounts of polymerization inhibitors, activators, color agents, and the like.

In certain embodiments, bone cement is prepared as described by U.S. Pat. No. 4,910,259 to Kindt-Larsen et al., which is incorporated herein in its entirety.

In those embodiments, the liquid component contain at least three distinct (meth)acrylate monomers. The three groups are listed below along with certain of the preferred materials:

(1) C₁-C₂ Alkyl methacrylates (e.g., methylmethacrylate and ethylmethacrylate);

(2) straight or branched long chain (meth)acrylates having a molecular weight of at least 168 and preferably 6 to 18 carbon atoms in the straight or branched chain substituents (e.g., n-hexylmethacrylate, n-heptylmethacrylate, ethylhexylmethacrylate, n-decylmethacrylate, isodecylmethacrylate, lauric methacrylate, stearic methacrylate, polyethyleneglycolmethacrylate, polypropyleneglycolmethacrylate, and ethyltriglycolmethacrylate); and

(3) Cyclic (meth)acrylates having a molecular weight of at least 168 and preferably 6 to 18 carbon atoms in the cyclic substituents (e.g., cyclohexymethacrylate, benzylmethacrylate, iso-bornylmethacrylate, adamantylmethacrylate, dicyclopentenyloxyethylmethacrylate, dicyclopentenylmethacrylate, dicyclopentenylacrylate, 3,3,5-trimethylcyclohexylmethacrylate, and 4-tert-butylcyclohexylmethacrylate).

As noted above, the liquid component or phase may contain crosslinking agents and minor amounts of additives such as polymerization inhibitors, activators, and the like. The polymerization inhibitors may be hydroquinone, hydroquinonemonomethylether, ascorbic acid, mixtures thereof, and the like in amounts ranging from about 10 to 500 ppm, preferably 20 to 100 ppm w/w. The activator is employed in amounts ranging from 0.2 to 3.0% w/w, preferably 0.4 to 1.0%, and may be N,N-dimethyl-p-toluidine, N,N-hydroxypropyl-p-toluidine, N,N-dimethyl-p-aminophen ethanol, N,N,-diethyl-p-aminophenyl acetic acid, and the like. It has been found helpful to use a combination of N,N-dimethyl-p-toluidine and N,N-hydroxypropyl-p-toluidine. Most preferably, the latter compound is used in greater proportions, e.g. 2 parts by weight for each part of N,N-dimethyl-p-toluidine. Useful crosslinking agents include ethyleneglycol dimethacrylate, 1,4-butanediol dimethacrylate, 1,3-butanediol dimethacrylate, triethyleneglycol dimethacrylate, tetraethyleneglycol dimethacrylate, polyethyleneglycol-400 dimethacrylate, neopentylglycol dimethacrylate, bisphenol A dimethacrylate, ethoxylated Bisphenol A dimethacrylate, trimethylolpropane trimethacrylate, and tripropyleneglycol acrylate.

The powder component or phase comprises a (meth)acrylate polymer, copolymer or a mixture of both. Illustrative materials include polyethylmethacrylate, polyisopropylmethacrylate, poly-sec-butylmethacrylate, poly-iso-butylmethacrylate, polycyclohexylmethacrylate, poly(butylmethacrylate-co-methylmethacrylate), poly(ethylmethacrylate-co-methylmethacrylate), poly(styrene-co-butylacrylate), and poly(ethylacrylate-co-methylmethacrylate).

The polymer powder may be utilized in finely divided form such as, for example, 20 to 250 microns. Admixed with the solid material may be X-ray contrast, polymerization initiator, antibiotics, antiseptic additives, and the like. Conventional X-ray contrast additives such as barium sulphate, zirconium dioxide, zinc oxide, and the like are used in amounts ranging from 5 to 15% w/w. Typical polymerization initiators can be used in amounts ranging from about 0.5 to 3.0% w/w. Examples of such initiators are benzoyl peroxide, lauroyl peroxide, methyl ethyl peroxide, diisopropyl peroxy carbonate. It will be understood that neither the use of most of the aforementioned additives nor the amounts thereof constitute essential features of the present invention. Moreover, the bone cement may also containing filler materials such as carbon fibers, glass fibers, silica, alumina, boron fibers, and the like.

The weight ratios of the liquid monomer component and the polymer powder component will range from between 1 to 1.5 and 1 to 2.5, preferably will be about 1 to 2.

As is well known in the art the final bone cement composition is obtained by mixing the liquid monomeric component with the free-flowing, polymeric powder component. The materials are admixed and dispensed in the conventional manner using known equipment.

Bone cement acts like a grout and not so much like a glue in arthroplasty. Although sticky, it primarily fills the spaces between the prosthesis and the bone preventing motion. It has a Young's modulus between cancellous bone and cortical bone. Thus, bone cement is a load sharing entity in the body without causing bone resorption.

Hydroxylapatite, also frequently called hydroxyapatite, is a naturally occurring form of calcium apatite with the formula Ca₅(PO₄)₃(OH), but is usually written Ca₁₀(PO₄)₆(OH)₂ to denote that the crystal unit cell comprises two molecules. The OH⁻ ion in the apatite group can be replaced by fluoride, chloride or carbonate. It crystallizes in the hexagonal crystal system. It has a specific gravity of 3.08 and is 5 on the Mohs hardness scale. Hydroxylapatite is the main mineral component of dental enamel, dentin, and bone.

Another example of a suitable matrix material is hydroxylapatite which can be used as a filler to replace amputated bone or as a coating to promote bone ingrowth into prosthetic implants. Although many other phases exist with similar or even identical chemical makeup, the body responds much differently to them. Coral skeletons can be transformed into hydroxylapatite by high temperatures; their porous structure allows relatively rapid ingrowth at the expense of initial mechanical strength. The high temperature also burns away any organic molecules such as proteins, preventing host vs. graft disease.

The term “a dental cement” or “a dental composite” as used herein, includes a composition which, after being cured, is stable and bonds well to hard tissues such as tooth enamel and dentin and to prostheses such as inlays, onlays, crowns, cores, posts and bridges that are formed of metals, porcelains, ceramics and composite resins, and which is therefore useful in restoring decayed or injured teeth and in bonding prostheses. An exemplary composition is described in U.S. Pat. No. 6,984,673 to Kawashima et al., which is incorporated herein in its entirety.

In certain embodiments, the moldable matrix is a thermoplastic polymer or a bone cement which further comprise a ceramic, a non-thermoplastic polymer or a combination thereof.

### The Method of Making Implant

In preparation of the implant of the invention, the selected moldable matrix and the magnetic/magnetizable objects are combined in a mold or a container to achieve a desired shape. The magnetic/magnetizable objects are optionally oriented prior to solidification or hardening of the matrix. The addition of the magnetic materials can occur by adding a dehydrated dispersion to the polymer powder, or by suspending the magnetic material within the monomer fluid. The mixing of the two agents and the beginning of the polymerization will allow for uniform mixing of the magnetic material within the matrix.

Conventional techniques of pressure molding or injection molding (e.g., U.S. Pat. No. 5,643,527) can be utilized for making implants prior to the implantation. Also, in situ formation of an implant can be utilized (see, for example, U.S. Pat. No. 5,945,115).

These combinations of moldable matrix and the magnetic/magnetizable objects of the invention may also be molded into infusion or injection devices, such as catheters, to provide antimicrobial solutions to blood pooling and microbial accumulation in vascular access devices, particularly for the treatment or ablation of hospital borne, drug-resistant microbes or other infectious agents.

During the molding of the matrix, permanent or electromagnetic assemblies may be used to align the magnetic material on the outer surface area or intraluminal surface area, depending on the geometry of the component, or uniformly distributed within the matrix of the component. In a preferred embodiment, the solidified product is formed from a moldable matrix having magnetic or magnetizable objects distributed in a pattern such that at least 50% of the magnetic or magnetizable objects are oriented along the outer surface of the implant.

Magnetic/magnetizable objects are added in the amount sufficient to enable producing heat upon the application of electromagnetic field. Concentrations may range from 1-25% by mass of magnetic or magnetizable material.

### The Method of Preventing or Eliminating an Infection

In another aspect, the invention is a method of preventing or eliminating an infection of an internal cavity of a mammal in the proximity of an implant, the method comprising: providing a moldable matrix; providing magnetic/magnetizable objects; combining the magnetic/magnetizable objects with the moldable matrix to form a composite and optionally orienting the magnetic/magnetizable objects within the moldable matrix; solidifying the composite and thereby forming a heat producing implant; administering the heat producing implant to the internal cavity of the mammal; activating the heat producing implant to prevent or eliminate the infection, wherein said activating is produced by applying an alternating magnetic field to the magnetic/magnetizable objects in the intensity sufficient to prevent or destroy infection producing microorganisms.

The majority of periprosthetic infections are caused by Staphylococcus aureus, and S. epidermidis, both gram-positive bacteria; less frequent infections are caused by the gram-negative organisms. Both S. aureus and S. epidermidis are commonly present in the operating room environment and are implicated in infections involving prostheses, stents and other implants. Both species adhere to the biomaterial surfaces, propagate rapidly, and during this proliferation, generate a pre-biofilm slime. Production of the polysaccharide-enclosed clumps of bacteria, characteristic of a biofilm, completes the process of restricting antibiotic access to the bacterial surface. This biofilm can effectively immobilize many antibiotics thereby reducing the numbers of therapeutic molecules that can penetrate and interact with the bacteria. Both S. aureus and S. epidermidis form such biofilms. When biofilms are formed, surgical experience dictates complete removal of the prosthetic components and debridement.

The infection can occur in an internal cavity of a mammal in the proximity of an implant within musculoskeletal tissue, connective tissue or a bone. For implants other than orthopedic implants, the infection can occur at the place of vascular access. For a partially implanted device, the infection may occur on the outer surface of the skin in the proximity of the implant.

Takegami et al. (1998; New Ferromagnetic Bone Cement for Local Hypethermia. John Wiley and Sons, Inc., 1998, 210-214) demonstrated heat generating ability of bone cement mixed with magnetite powder upon application of alternating magnetic field where the temperature of the interface between the bone and the surrounding muscle reached 43-45° C. when the temperature in the implanted thermoseed was maintained at 50-60° C.

By applying an alternating magnetic field to the magnetic/magnetizable objects, temperatures just above 37° C. and higher are generated for ablation or denaturing of bacteria, fungal, or other microbial contamination at the site of the implant. Non-limiting examples of devices or generators of alternating magnetic field include those described in U.S. Patent Application Publication No. 2006/0009826 to Gleich.

High frequency alternating magnetic fields can be produced using electromagnets powered by AC Power supplies, as high as 5 kw, or as low as 0.1 kW. This can be produced as a handheld, bedside, or C-Arm device, and with low magnetic field magnitude, no magnetic shielding of the room will be required.

The method of the invention can be practiced as a prophylactic measure to prevent the infection at the time or immediately after the implantation. This can be done in a hospital setting by a healthcare professional or at home by a utilizing a portable AC Power supply.

Also, if the infection has occurred, the method of the invention can be practiced as a treatment measure to eliminate the infection. Monitoring of the progress can be done by blood test or monitoring temperature of the patient.

Additionally, traditional methods of treating infections such as administering antibiotics can be combined with the method of the invention.

Hospital born infections of drug resistant microbes, can be treated or prevented using this method, or paired with antibiotic therapies.

In another aspect, the invention is a method of treatment of a local area of an implant to prevent or treat loosening of the implant. In the case of the loosening, this means that there is a reduction in bone tissue mass around the implant because of a loading mismatch over the course of the joint implant's lifetime. It is believed that by heating the area, slight necrosis (tissue death) can be induced in the nearby bone, causing a rapid scar response to spur a return to proper bone-implant fixation. Thus, the method can be practiced by activating the heat producing implant to prevent or treat loosening of the implant, wherein said activating is produced by applying an alternating magnetic field to the magnetic/magnetizable objects in the intensity sufficient to prevent or treat loosening of the implant.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A heat producing implant comprising a solidified product of a moldable matrix having magnetic or magnetizable objects distributed in a pattern such that at least 50% of the magnetic or magnetizable objects are oriented along the surface of the implant, wherein the heat producing implant is capable of generating a controllable heat upon application of controlled alternating magnetic fields in the intensity sufficient to destroy infection producing microorganisms.

2. The heat producing implant of claim 1, wherein the moldable matrix comprises a thermoplastic polymer.

3. The heat producing implant of claim 1, wherein the moldable matrix comprises a bone cement.

4. A method of preventing or eliminating an infection of an internal cavity of a mammal in the proximity of an implant, the method comprising:
providing a moldable matrix;
providing magnetic/magnetizable objects;
combining the magnetic/magnetizable objects with the moldable matrix to form a composite and optionally orienting the magnetic/magnetizable objects within the moldable matrix;
solidifying the composite and thereby forming a heat producing implant;
administering the heat producing implant to the internal cavity of the mammal;
activating the heat producing implant to prevent or eliminate the infection, wherein said activating is produced by applying an alternating magnetic field to the magnetic/magnetizable objects in the intensity sufficient to prevent or destroy infection producing microorganisms.

5. The method of claim 4, wherein said moldable matrix comprises a thermoplastic polymer.

6. The method of claim 4, wherein said moldable matrix comprises a bone cement.
